(19) United States
(12) Patent Application Publication
(10) Pub. No.: US 2002/0015734 A1
(43) Pub. Date: Feb. 7, 2002

(54) Thermoreversible polymers for delivery and retention of osteoinductive proteins

(75) Inventors: Hasan Uludag, Edmonton (CA); Tiejun Gao, Edmonton (CA); John Wozney, Hudson, MA (US)

Correspondence Address: AMERICAN HOME PRODUCTS CORPORATION, FIVE GIRALDA FARMS, PATENT LAW, MADISON, NJ, 07940, US

(73) Assignee: Genetics Institute, Inc., Cambridge, MA

(21) Appl. No.: 09/814,543

(22) Filed: Mar. 22, 2001

Related U.S. Application Data
(60) Provisional application No. 60/191,533, filed on Mar. 23, 2000.

(51) Int. Cl. A61K 38/43; A61K 9/14

(52) U.S. Cl. 424/486; 424/94.1

(57) ABSTRACT

A temperature-sensitive polymer formulation for delivery of osteoinductive proteins is disclosed. The formulation comprises a pharmaceutically acceptable admixture of a temperature sensitive polymer and an osteoinductive protein. The formulations of the present invention enhance the retention of the osteoinductive protein at the site of administration.

This application claims priority from copending provisional application Ser. No. 60/191,533 filed on Mar. 23, 2000.

The subject invention relates to the delivery of osteoinductive proteins. More particularly, the subject invention is directed to the delivery of osteoinductive proteins using temperature sensitive polymers which enhance retention of the protein.

Osteogenic proteins are those proteins capable of inducing, or assisting in the induction of, cartilage and/or bone formation. Many such osteogenic proteins have in recent years been isolated and characterized, and some have been produced by recombinant methods. The osteogenic proteins useful with the thermoreveersible polymers made in accordance with the subject invention are well known to those skilled in the art and include those discussed above. For example, so-called bone morphogenic proteins (BMP) have been isolated from demineralized bone tissue (see e.g. Urist U.S. Pat. No. 4,455,256); a number of such BMP proteins have been produced by recombinant techniques (see e.g. Wang et al. U.S. Pat. No. 4,877,864 and Wang et al. U.S. Pat. No. 5,013,549); a family of transforming growth factors (TGP-α and TGF-β) has been identified as potentially useful in the treatment of bone disease (see e.g. Derynck et al., EP 154,434); a protein designated Vgr-1 has been found to be expressed at high levels in osteogenic cells (see Lyons et al. (1989) Proc. Nat'l. Acad. Sci. USA 86, 4554-4558); and proteins designated OP-1, COP-5 and COP-7 have purportedly shown bone inductive activity (see Oppermann, et al. U.S. Pat. No. 5,001,691).

Various formulations designed to deliver osteogenic proteins to a site where induction of bone formation is desired have been developed. Although certain BMPs and in particular BMP-2 is capable of inducing de novo bone formation by itself, a suitable delivery system typically augments the rhBMP-2 bioactivity, defines three dimensional geometry for bone in growth and improves the reproducibility of osteoinduction. For example, certain polymeric matrices such as acrylic ester polymer (Urist, U.S. Pat. No. 4,526,909) and lactic acid polymer (Urist, U.S. Pat. No. 4,563,489) have been utilized. A biodegradable matrix of porous particles for delivery of an osteogenic protein designated as OP is disclosed in Kuberasampath, U.S. Pat. No. 5,108,753. Brekke et al., U.S. Pat. Nos. 4,186,448 and 5,133,755 describe methods of forming highly porous biodegradable materials composed of polymers of lactic acid (“OPLA”). Okada et al., U.S. Pat. No. 4,652,441, No. 4,711,782, No. 4,917,893 and No. 5,061,492 and Yamamoto et al., U.S. Pat. No. 4,954,298 disclose a prolonged-release microcapsule comprising a polypeptide drug and a drug-retaining substance encapsulated in an inner aqueous layer surrounded by a polymer wall substance in an outer oil layer. Yamazaki et al., Clin. Orthop. and Related Research, 234:240-249 (1988) disclose the use of implants comprising 1 mg of bone morphogenetic protein purified from bone and 5 mg of Plaster of Paris. U.S. Pat. No. 4,645,503 discloses composites of hydroxyapatite and Plaster of Paris as bone implant materials. Collagen matrices have also been used as delivery vehicles for osteogenic proteins (see e.g. Jeffries, U.S. Pat. No. 4,394,370).

### SUMMARY OF THE INVENTION

The present invention provides temperature sensitive formulations for the delivery of osteogenic proteins. The polymers are designed to provide a novel mechanism for in situ retention of osteoinductive protein. In one embodiment, the invention comprises compositions comprising a pharmaceutically acceptable admixture of an osteogenic protein together with a formulation of a thermoreversible polymer (i.e. polymers that exhibit temperature sensitive solubility). Temperature sensitive polymers exhibit a controlled phase transformation from a soluble to an insoluble state. The thermoreversible feature of the polymers allows one to carry out desired manipulations in a solution phase but eventually to induce a solid phase upon exposure to a temperature above the solubility limit of the polymers. Being insoluble at physiological temperature these polymers sequester the proteins at a site of administration. Thermoreversible polymers enhance healing in defects by enhancing retention of the osteoinductive protein at the local site. In a preferred embodiment, the formulation comprises osteogenic protein and temperature-sensitive polymer based on N-isopropylacrylamide (NiPAM). In a further preferred embodiment ethyl methacrylate (EMA) and N-acryloxysuccinimide (NASI) are incorporated into the NiPAM polymer to reduce the lower critical solution temperature (LCST) and to allow conjugation to proteins. In a further embodiment alkyl methacrylate (AMA) other than EMA may be incorporated such as butylmethacrylate (BMA), hexylmethacrylate (HMA) and dodecylmethacrylate (DMA).

The methods and compositions of the present invention are useful for the preparation of formulations of osteoinductive proteins which can be used, among other uses, to promote the formation of cartilage and/or bone, for repair of tissue damage and fractures. The invention further provides methods for treating patients in need of cartilage and/or bone repair and/or growth. The compositions of the invention may be injected or implanted.

A further embodiment of the invention is directed to thermoreversible polymers for the delivery of therapeutic agents.

### BRIEF DESCRIPTION OF THE DRAWINGS

FIG. 1 sets forth LCST (A) and water uptake (B) of NiPAM (o), NiPAM/NASI (♦) and NiPAM/EMA (n) copolymers. NiPAM homopolymer exhibited a higher LCST (26.7° C.) compared to NiPAM-NASI (18.5° C.) and NiPAM-EMA copolymers (19.−° C.). NiPAM-EMA gels were more stable than NiPAM gels. NiPAM/NASI were not able to form gels (not shown).

FIG. 2 sets forth in vitro rhBMP-2 retention in collagen sponges (A) and polymer gels (B). The sponge retention of rhBMP-2 in the presence of a polymer (3.9 mg/mL) was initially lower but subsequent release was relatively similar among the groups. In the absence of a sponge, only B30% of rhBMP-2 was released into the medium, indicating that rhBMP-2 was not readily soluble in SBF release medium. NiPAM/NASI released the protein faster after 72 hours most likely due to polymer hydrolysis.

FIG. 3 sets forth in vivo retention profiles for rhBMP-2 delivered with or without the polymers. (A) Implantation with a collagen sponge using a polymer concentration of 3.9 mg/mL. (B) Implantation with a collagen sponge using a polymer concentration of 28.7 mg/mL. (C) Injection with a polymer concentration of 28.7 mg/mL. Note that the injectable format using polymers NiPAM/NASI and NiPAM/EMA gave the highest in situ retention.

FIG. 4 sets forth the compositions and the LCsTs of the polymers selected for reactivity with rhBMP-2.

FIG. 5 (A) Mean ±SD percent retention of rhBMP-2 at the injection site after 1, 7 and 14 days. The polymers used in this study were NiPAM/BMA or NiPAM/BMA/NASI at a relatively low and high LCST (see legend). Note that NiPAM/BMA with a low LCST, as well as NiPAM/BMA/NASI polymers (irrespective of LCST) gave a significantly higher localization of the protein after 7 and 14 days. (B) Percent retention of rhBMP-2 at the injection site after 14 days using HMA containing polymers. The rhBMP-2 retention was again the highest for the NASI containing polymers, followed by the polymer with low LCST.

### DETAILED DESCRIPTION OF THE INVENTION

The present invention provides thermoreversible polymer compositions for the delivery of osteogenic proteins. The compositions comprise osteogenic protein and an injectable or implantable formulation includes the osteogenic protein, the formulation of temperature-sensitive polymer and a carrier. The invention further provides a method for preparing the temperature-sensitive polymer and the invention includes the composition prepared by this method. Biomaterials play a critical role for the therapeutic delivery of osteoinductive proteins. Biomaterials may provide a three dimensional template into which cell migration takes place. A cell-compatible biomaterial helps support cell proliferation and, by providing a suitable attachment substrate, may directly influence cellular transformation into the differentiated osteogenic phenotype. A biomaterial may additionally present the osteoinductive protein to the infiltrating cell type in an appropriate fashion. It is contemplated that rhBMP binding to a biomaterial helps to localize the protein at a site of application.

Synthetic polymers of the invention may be selected by those skilled in the art based on the desired physicochemical characteristics which will ultimately control the protein delivery. One such characteristic, lower critical solution temperature (LCST), has been identified as critical, since the polymers are desired to be formulated as aqueous solutions for injection, but to be insoluble once delivered to the treatment site. Temperature dependent solubility was ideal for this purpose, since no exogenous agent is needed to induce the required phase transformation. Thermoreversible polymers have been prepared, most commonly from N-isopropylacrylamide (NiPAM), and demonstrated a predictable polymer LCST based on the polymer composition [see for example, Chem. Phys. (1999) 200:51-57; and Macromol. (1998) 5616-5623 (1998)]. In one embodiment polymers are synthesized from the base monomer of NiPAM and comonomers EMA and NASI. The polymers were based pm N-isopropylacrylamide (NiPAM). NiPAM-based polymers are compatible with the osteoinductive activity of the rhBMP-2. Ethyl methacrylate (EMA) and N-acryloxysuccinimide (NASI) were incorporated into the NiPAM polymer to reduce the lower critical solution temperature and to allow conjugation to proteins, respectively. Three polymers distinct in their characteristics, a NiPAM homopolymer (LCST ˜27° C.), a NiPAM/ethyl methacrylate copolymer (NiPAM/EMA; LCST: ˜19° C.), and a protein reactive NiPAM/N-acryloxysuccinimide copolymer (NiPAM/NASI; LCST ˜19° C.) have demonstrated compatibility with rhBMP-2 induced de novo bone formation in a rat ectopic implant model. Temperature sensitive formulations of the invention possess the advantages of enhancing retention of the osteoinductive protein at the delivery site. Increased retention is expected to increase the effectiveness of osteogenic proteins to induce de novo bone.

A change in MW of synthesized polymers, irrespective of the presence of a NASI group, alters the hydrogel structure and stability in vitro. The MW effect on rhBMP-2 retention depends on the type of polymer: whereas the performance of polymers designed for chemical conjugation appears insensitive to MW, the performance of polymers designed for physical entrapment is significantly affected by the polymer MW. Using different synthetic approaches, one skilled in the art can engineer the properties of thermoreversible polymers and alter the therapeutic protein retention in order to meet different treatment modalities for which therapeutic protein is being explored.

The osteogenic proteins useful with the thermoreveersible polymers made in accordance with the subject invention are well known to those skilled in the art. The preferred osteogenic proteins for use herein are those of the BMP class which have been disclosed to have osteogenic, chondrogenic and other growth and differentiation type activities. These BMPs include rhBMP-2, through BMP-12, rhBMP-13, rhBMP-15, rhBMP-16, rhBMP-17, rhBMP-18, rhGDF-1, rhGDF-3, rhGDF-5, rhGDF-6, rhGDF-7, rhGDF-8, rhGDF-9, rhGDF-10, rhGDF-11, rhGDF-12, rhGDF-14. For example, BMP-2, BMP-3, BMP-4, BMP-5, BMP-6 and BMP-7, disclosed in U.S. Pat. Nos. 5,108,922; 5,013,649; 5,116,738; 5,106,748; 5,187,076; and 5,141,905; BMP-8, disclosed in PCT publication WO91/18098; and BMP-9, disclosed in PCT publication WO93/00432, BMP-10, disclosed in U.S. Pat. No. 5,637,480; BMP-11, disclosed in U.S. Pat. No. 5,639,638, or BMP-12 or BMP-13, disclosed in U.S. Pat. No. 5,658,882, BMP-15, disclosed U.S. Pat. No. 5,635,372 and BMP-16, disclosed in co-pending patent application Ser. No. 08/715,202. Other compositions which may also be useful include Vgr-2, and any of the growth and differentiation factors [GDFs], including those described in PCT applications WO94/15965; WO94/15949; WO95/01801; WO95/01802; WO94/21681; WO94/15966; WO95/10539; WO96/01845; WO96/02559 and others. Also useful in the present invention may be BIP, disclosed in WO94/01557; HP00269, disclosed in JP Publication number: 7-250688; and MP52, disclosed in PCT application WO93/16099. The disclosures of all of these applications are hereby incorporated herein by reference. Of course, combinations of two or more of such osteogenic proteins may be used, as may fragments of such proteins that also exhibit osteogenic activity. Such osteogenic proteins are known to be homodimeric species, but also exhibit activity as mixed heterodimers. Heterodimeric forms of osteogenic proteins may also be used in the practice of the subject invention. BMP heterodimers are described in WO93/09229, the disclosure of which is hereby incorporated by reference. Recombinant proteins are preferred over naturally occurring isolated proteins. These proteins can be used individually or in mixtures of two or more, and rhBMP-2 is preferred.

The amount of osteogenic protein useful herein is that amount effective to stimulate increased osteogenic activity of infiltrating progenitor cells, and will depend upon the size and nature of the defect being treated, as well as the carrier being employed. Generally, the amount of protein to be delivered is in a range of from about 0.05 to about 1.5 mg.

The invention further provides a method for treating a patient in need of the induction of cartilage and/or bone formulation. The therapeutic method includes administering and composition, systematically, by injection or locally as an implant or device. Injectable formulations may also find application to other bone sites such as bone cysts and closed fractures. The injectable osteogenic protein may be provided to the clinic as a single formulation, or the formulation may be provided as a multicomponent kit. When administered, the therapeutic composition for use in this invention is, of course, in a pyrogen-free, physiologically acceptable form. Further, the composition may desirably be encapsulated or injected in a viscous form for delivery to the site of cartilage and/or bone or tissue damage. Topical administration may be suitable for wound healing and tissue repair. Preferably for bone and/or cartilage formation, the composition includes a matrix capable of delivering the cartilage/bone proteins of the invention to the site of bone and/or cartilage damage, providing a structure for the developing bone and cartilage and optimally capable of being resorbed into the body. Matrices may provide slow release of the cartilage and/or bone inductive proteins proper presentation and appropriate environment for cellular infiltration. Matrices may be formed of materials presently in use of other implanted medical applications. The selection of the carrier is within the knowledge of those skilled in the art. Such carriers include collagen derivatives including collagen sponges.

The BMP may be recombinantly produced, or purified from a protein composition. The BMP may be homodimeric, or may be heterodimeric with other BMPs (e.g., a heterodimer composed of one monomer each of BMP-2 and BMP-6) or with other members of the TGF-β superfamily, such as activins, inhibins and TGF-β1 (e.g., a heterodimer composed of one monomer each of a BMP and a related member of the TGF-β superfamily). Examples of such heterodimeric proteins are described for example in Published PCT Patent Application WO 93/09229, the specification of which is hereby incorporated herein by reference.

The formulations of the invention may be injected or implanted. Injectable formulations may also find application to other bone sites such as bone cysts and closed fractures.

The dosage regimen will be determined by the clinical indication being addressed, as well as by various patient variables (e.g. weight, age, sex) and clinical presentation (e.g. extent of injury, site of injury, etc.). In general, the dosage of osteogenic protein will be in the range of from about 0.1 to 4 mg/ml.

The injectable osteogenic protein may be provided to the clinic as a single formulation, or the formulation may be provided as a multicomponent kit.

The formulations of the subject invention allow therapeutically effective amounts of osteoinductive protein to be delivered to an injury site where cartilage and/or bone formation is desired. The formulations may be used as a substitute for autologous bone graft in fresh and non-union fractures, spinal fusions, and bone defect repair in the orthopaedic field; in cranio/maxillofacial reconstructions; in osteomyelitis for bone regeneration; and in the dental field for augmentation of the alveolar ridge and periodontal defects and tooth extraction sockets. The methods and formulations of the present invention may be useful in the treatment and/or prevention of osteoporosis, or the treatment of osteoporotic or osteopenic bone. In another embodiment, formulations of the present invention may be used in the process known as distraction osteogenesis. When used to treat osteomyelitis or for bone repair with minimal infection, the osteogenic protein may be used in combination with porous microparticles and antibiotics, with the addition of protein sequestering agents such as alginate, cellulosics, especially carboxymethylcellulose, diluted using aqueous glycerol. The antibiotic is selected for its ability to decrease infection while having minimal adverse effects on bone formation. Preferred antibiotics for use in the devices of the present invention include vancomycin and gentamycin. The antibiotic may be in any pharmaceutically acceptable form, such as vancomycin HCl or gentamycin sulfate. The antibiotic is preferably present in a concentration of from about 0.1 mg/mL to about 10.0 mg/mL.] The traditional preparation of formulations in pharmaceutically acceptable form (i.e. pyrogen free, appropriate pH and isotonicity, sterility, etc.) is well within the skill in the art and is applicable to the formulations of the invention.

To test the capacity of polymers to retain rhBMP-2, rhBMP-2 was labeled with ¹²⁵I. Formulated with the polymers and was either implanted with a collagen sponge or injected directly into an intramuscular site in rats. The results indicated that implantation with a relatively low polymer concentration (3.9) mg/mL did not result in significant rhBMP-2 retention, but increasing the polymer concentration (28.7 mg/mL) gave a better retention with NiPAM/NASI polymers. Synthetic, temperature-sensitive polymers can be engineered to sequester and retain osteoinductive proteins at a site of administration. These biomaterials may allow to development of osteoinductive products with enhancement potency.

The following examples further describe the practice of embodiments of the invention with tempersture sensitive polymers and BMP-2. The examples are not limiting, and as will be appreciated by those skilled in the art. Modifications, variations and minor enhancements are contemplated and are within the present invention and within the knowledge of those skilled in the art.

### EXAMPLE 1

Materials

rhBMP-2 was produced in CHO cells transfected with a pMT2 expression vector [Grow. Fac. 7:139-150 (1992).] and formulated in a glycine buffer containing 0.5% Sucrose, 2.5% Glycine, 5 mM Glutamic Acid, 5 mM NaCl and 0.01% Tween-80 (pH 4.5) under cGMP conditions (Lot PC4579-135, 4.4 mg/mL). The rhBMP-2 solution was buffer-exchanged into 0.1 MMES buffer. ¹²⁵I was from Amersham (Baie d'Urfé, Quebec) and used to label rhBMP-2 with Iodo-Gen® reagent (Pierce; Rockford, Ill.). Absorbable Helistat® collagen sponge was from Integra Life Sciences, (Plainsboro, N.J.). The sources of all monomers and various chemical reagents are set forth in Fang and Uludag Drug Delivery in the 21st Century (1999) ACS. Simulated body fluid (SBF: 142.0 mM Na*, 5.0 mM K⁺2.5 mM Ca⁺² 1.5 mM MG⁺², 147.8 mM Cl, 4.2 mM HCO₃; 1.0 mM HPO₄⁻², 0.5 mM SO₄⁻²) was prepared according to Kokubo et al., J. Biomed. Mat. Res. (1990)24: 721-734. Female Sprague-Dawley rats aged 4 to 6 weeks with body weight 200-250 grams were supplied by Biosciences (Edmonton, AB).

### EXAMPLE 2

Polymer Synthesis and Characterization

The preparation of NiPAM-based thermoreversible polymers is set forth in Fan and Uludag Drug Delivery in the 21st Century (2000) ACS. A desired amount of NiP AM, NASI or EMA was dissolved in dioxane, the free radical initiator benzoylperoxide was then added to this solution and the polymerization was performed at 70° C. for 22 hours under a N₂ blanket. The polymers were precipitated by hexane and compositions were determined by proton NMR.

To determine polymer LCST, 10 mg/mL polymer solutions (in 0.1 M phosph ate buffer, pH—7.4) were placed in a spectrophotometer equipped with a water-circulation chamber [Fan and Uludag Drug Delivery in the 21st Century (1999) ACS]. The optical density (O.D.) at 420 nm vs. temperature curves were fitted with a sigmoidal curve and temperature at the inflexion point was taken as the LCST. The stability of the polymer hydrogels was also evaluated as a function of temperature [Fan and Uludag Drug Delivery in the 21st Century (1999) ACS.] Dry polymer films were immersed in 0.1 M phosphate buffer (pH 7.4) at 35° C. and the temperature was slowly lowered until the hydrogels were dissolved. The water uptake of the films was calculated at specific temperatures by: (wet weight/dry weight)×100%.

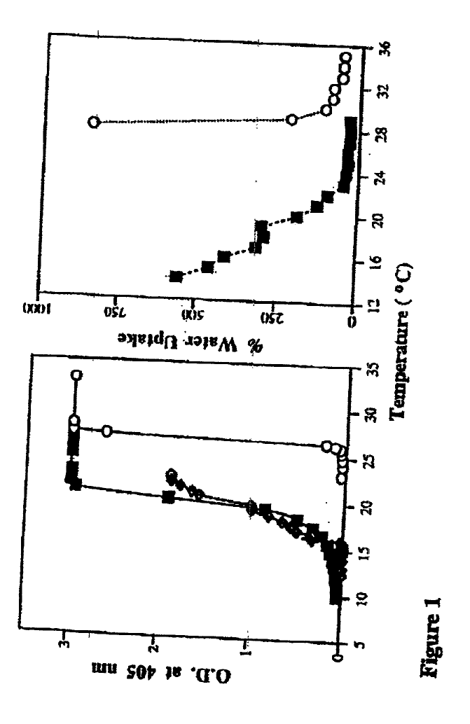

The final polymer composition was effectively controlled by the monomer feed ratios during polymerization [(Fang and Uladag Drug Delivery in the Twentieth Century (1999) ACS Washington, D.C.]. From a series of NiPAM, NiPAM/EMA and NiPAM/NASI copolymers, a NiPAM homopolymer and copolymers with EMA and NASI contents of 26/3% (feed: 15/4%) and 7.2% (feed: 9.1%), were chosen, respectively. The LCST of NiPAM homopolymer was 26.7° C., whereas the LCST of NiPAM/EMA and NiPAM/NASI were 19.4° C. and 18.5° C. (FIG. 1A), respectively. To determine whether the polymers were able to form gels, 10 mg/mL polymer solutions were heated up from 10° C. to 37° C. at 1° C./day. The NiPAM solution turned cloudy at 27° C. in accordance with the LCST but formed a small (<10% of solution volume) gel above LCST. The NiPAM/NASI exhibited a turbidity after 29° C. (considerably higher than the LCST) and did not form gels at all. The NiPAM/EMA exhibited turbidity at 19° C. and formed a solid gel at 31° C. In a modification of this study, the water uptake of polymer films as a function of temperature is shown in FIG. 1B. The NiPAM film was stable above 27° C., NiPAM/EMA was stable at a temperature as low as 14° C., but NiPAM/NASI film was dissolved immediately after being immersed in the phosphate buffer (pH=7.4).

The conjugation reaction between rhBMP-2 and polymers was investigated by mixing a polymer solution (in phosphate buffer) with a rhBMP-2 solution (in MES buffer) at 4° C. After a specific period of incubation, the reaction was quenched by glycine buffer and the solution was loaded onto 4-15% SDS-PAGE gels. The gels were stained with 0.025% Coomassie blue for 6-8 hours and destained with 10% isopropanol-acetic acid. The conjugation was assessed by disappearance of native rhBMP-2 band (˜32 kD) and appearance of high molecular weight species, consistent with high molecular weight of the polymer (100-200 kD).

In some studies to ensure that disappearance of rhBMP-2 band correspond to rhBMP-2 conjugation, a western immunoblot of the electrophoresed proteins was carried out. The proteins were transferred to a nitrocellulose membrane using Mini Trans-Blot (Bio-Rad) at 300 mA for 1.5 hours in a buffer containing 191 mM glycine, 25 mM Tris, 20% methanol and 0.05% SDS. After washing and blocking with 4% BSA, the membrane was incubated with h3b2/17.8.1 monoclonal antibody (1 μg/ml) in the blotting buffer for 3 hours at room temperature. The membrane was then incubated with the alkaline phosphatase-conjugated goat anti-mouse IgG (1:1500 dilution) for 2 hours at room temperature, and the antibody-reactive bands were visualized by BCIP-NIP.

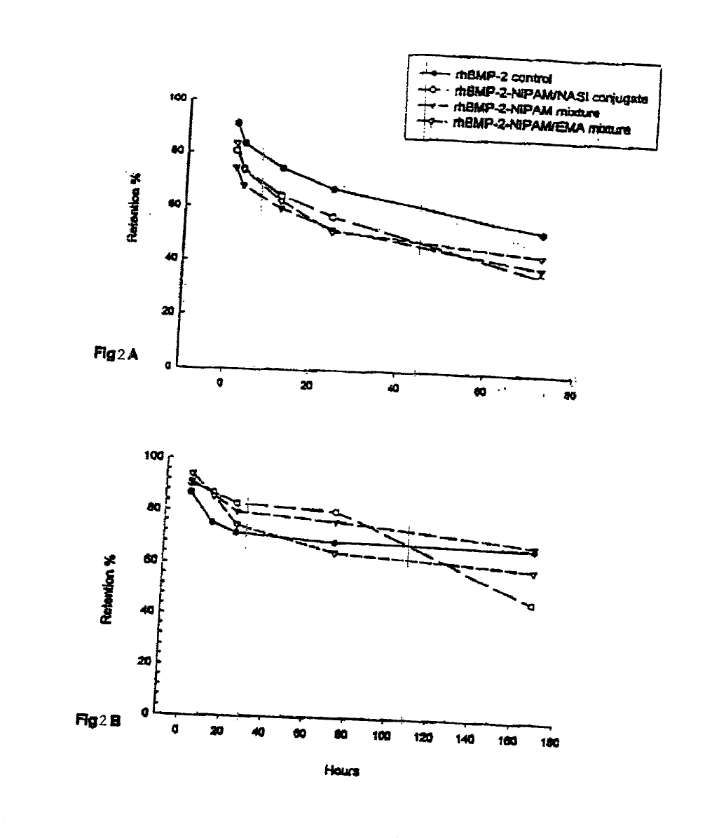

Based on SDS-PAGE analysis, NiPAM/NASI reacted with rhBMP-2 but no reaction was seen with NiPAM and NiPAM/EMA. The conjugation efficiency (as assessed by disappearance of native rhBMP-2 band and appearance of high MW protein species on gels) was correlated with the incubation time: little reaction was seen after 15 minutes whereas a complete conjugation was obtained after 6 hours of incubation. The conjugation efficiency was proportional to the relative concentration of NiPAM/NASI to rhBMP-2. Some conjugation was observed at a polymer:rhBMP-2 ratios of 40:1 (based on concentration ratios) whereas an apparently complete conjugation reaction was obtained at polymer:rhBMP-2 ratios 80:1 and 128:1 after 3 hour reaction (FIG. 2). Consequently, the following in vitro release and in vivo PK studies were conducted using a rhBMP-2: polymer ratio of at least 130:1.

### EXAMPLE 3

Formulation of rhBMP-2 for Implantation and Injection

The rhBMP-2 solution used for pharmacokinetics studies was obtained by adding a trace amount of ¹²⁵I-rhBMP-2 to unlabeled rhBMP-2 solution (hot:cold rhBMP-2±1:160). The ¹²⁵I-labeling was performed according to a previous report [J. Biomed. Mat. Res. (1999) 46:193-202], except that MES buffer was used during labeling instead of the glycine buffer. This was necessary since the presence of amino acids in glycine buffer interferes with the subsequent polymer conjugation reaction. Precipitation of labeled rhBMP-2 with 20% trichloroacetic acid (TCA) gave >98% precipitable (i.e., protein-bound) counts.

A separate rhBMP-2 iodination was performed for each of the 3 different animal studies, two implantations and one injection (see Table 1 for the design of overall study). In the first implant study, rhBMP-2 solution (2.4 mg/ML) was incubated with a polymer solution (30 mg/mL in 0.1 M phosphate buffer) for 3 hours at 4° C. The mixture was then diluted with glycine buffer to give final rhBMP-2 and polymer concentrations of 30 μg/mL 3.9 mg/mL, respectively (1:130 rhBMP-2:polymer ratio). In the second implant study, the rhBMP-2 solution was incubated with polymer solutions in the same way, except it was diluted with a glycine buffer that contained 30 mg/mL polymer, giving a final polymer concentration of 28.7 mg/mL (1:950 rhBMP-2:polymer ratio). The polymer concentration in the injection study was the same as the second implant study.

### EXAMPLE 4

Implantation and Injection Procedures.

Collagen implants were 14×14 mm squares, cut from 3″×4″ Helistat® sponge (3.5 mm thickness). 200 μL of radioactive rhBMP-2 solution was added to all implants in sterile 100 mm petri dishes and allowed to soak for at least 10 minutes before implantation. A minimum of one week acclimatization period was allowed between the receipt of the Sprague-Dawley rats and the start of the study to allow animals to adjust to the new environment. The animals were anaesthetized with methoxyflurane inhalation (JANSSEN Pharmaceutical, ON, CA). After scrubbing the implantation area with liberal amounts of Betadine, two 4 mm incisions were made on each side of hind leg. An intramuscular pouch was created with tissue scissors in the gluteus meximus and sponges were inserted into the pouch. Opening of the pouch was closed by one stitch of 5-0 polyethylene suture and skin incision was closed with staples. The animals were watched until they regained consciousness.

Injections of rhBMP-2/polymer formulations were performed on anaesthetized rats. All solutions were kept at 4° C. until injection time. A small skin incision (2-3 mm) was made to ensure accurate injection into the compartment of the gluteus maximus of hind leg 100 μL of solution was directly injected into the both muscle sites of rats using an insulin syringe and the skin incision was closed with staples. The animals were watched until they regain consciousness.

### EXAMPLE 5

RhBMP-2 Recovery and Pharmacokinetics (PK) Analysis

At indicated time points, 2 rats from each group were sacrificed (4 implants per time point) by Euthany (MTC Pharmaceuticals, Cambridge, Ontario) injection and the implants or muscle tissue injected with the protein was retrieved. The radioactivity associated with implants was determined by a y-counter (Wizard 1470; Wallace Inc., Turku, Finland) at the time of retrieval. The muscle around the implant was also recovered and counted to determine the rhBMP-2 in the implant vicinity. In the case of injection, gluterus maximus containing injected solutions was harvested en bloc at designated time-points and counted as a whole. Previous studies indicated that most counts (>90%) were precipitable with TCA, so that TCA-precipitation was not performed in this study.

To visualize in vivo distribution of ¹²⁵I-labeled rhBMP-2, the gluteus maximus samples retrieved at 1 and 5 days after injection were exposed to Kodak X-OMAT high resolution film at 4° C. for 1 week. The distribution patterns and intensity of the blotting images on the films were compared among different polymers-rhBMP-2 mixtures and conjugates.

The radioactive counts in explants were used as a measure of rhBMP-2 in the implants. All counts were corrected for radioactive decay by assuming ¹²⁵I half-lives of 60 days and are shown as time=0 (designated as implantation time) counts. The rate of rhBMP-2 loss from the implants was analyzed non-compartmentally by the trapezoid-rule. The percent retention vs. time curves were generated by dividing the recovered radioactive counts by the counts originally implanted or injected. Non-compartmental analysis was used to calculate areas under the curve (AUC), areas under the moment curve (AUMC) and mean residence time (MRT=AUMC+AUC).

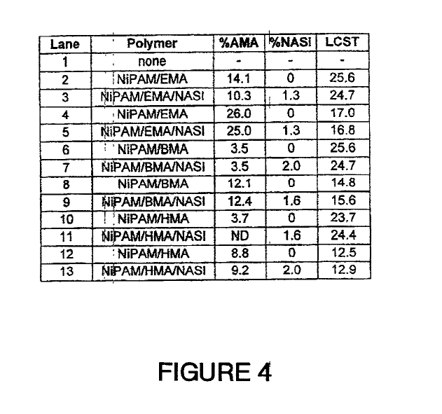

The results of the first implant study where polymer concentration was 3.9 mg/mL are summarized in FIG. 4A. Compared to rhBMP-2 control, group, none of the polymers gave a significantly different retention on either day 1, day 5 or day 9. The AUC or MRT for the control rhBMP-2 was also not different from that of the polymer groups (Table 1). The second implant study was carried out by increasing the polymer concentration to 28.7 mg/mL (FIG. 4B). There was no difference in rhBMP-2 retention on the day 1 among the study groups. Day 5 and day 9 rhBMP-2 retention for NiPAM/NASI groups was higher than the control rhBMP-2 groups, but no difference was obtained with the other polymers. The AUC for NiPAM and NiPAM/NASI group was significantly higher than the control rhBMP-2, but MRT was not different among these groups.

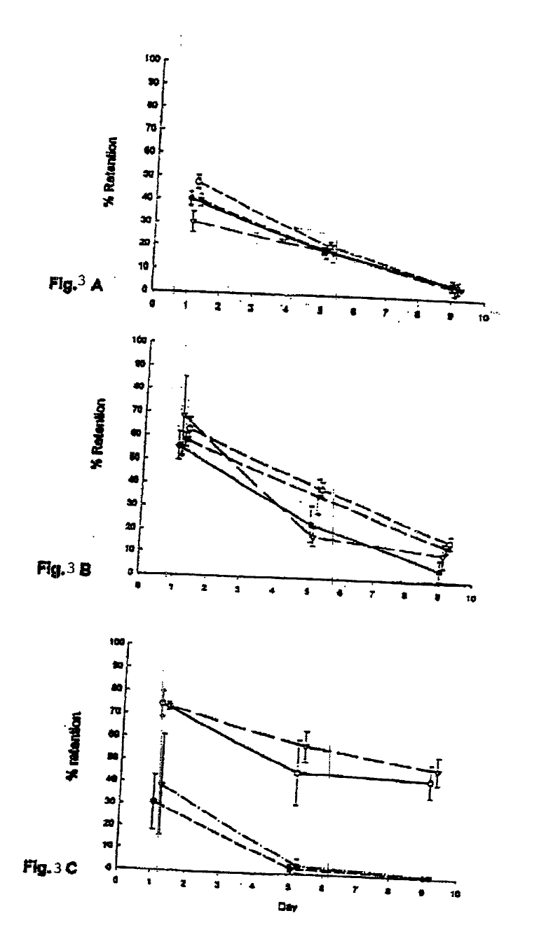

In the injection study (FIG. 3C), significant differences among the study groups were evident on days 1, 5 and 9. Whereas NiPAM and control rhBMP-2 had equivalent retention. Day 1, NiPAM/EMA and NiPAM/NASI gave a ˜2 fold increased rhBMP-2 retention. The subsequent rhBMP-2 loss from the NiPAM and rhBMP-2 control group was similar and rapid. However, little rhBMP-2 loss was observed from NiPAM/EMA and NiPAM/NASI in the subsequent days. On day 5 and 9, these two polymers gave 17-21, fold and 218-242 fold higher rhBMP-2 retention, respectively. Consistent with this AUC and MRT for NiPAM/EMA and NiPAM/NASI groups were significantly higher than the groups where NiPAM or no polymer was injected (Table 1).

TABLE 1
Study Groups for In Vivo Delivery and Calculated Pharmacokinetic Parameters

| | | Delivery | Sample | rhBMP-2 | Polymer | | | |
|---|---|---|---|---|---|---|---|---|
| Study | Composition | Method | Volume | Dose | Concentration | Sacrifice | AUC | MRT |
| 1 | rhBMP-2 | Implantation | 200 μl. | 6 μg | 3.9 mg/ml. | 2 rats at 1, 5 and 9 days | 170.6 | 3.4 days |
| | rhBMP-2 + NiPAM | Implantation | 200 μl. | 6 μg | 3.9 mg/ml. | 2 rats at 1, 5 and 9 days | 162.6 | 3.3 days |
| | rhBMP-2 + NiPAM/EMA | Implantation | 200 μl. | 6 μg | 3.9 mg/ml. | 2 rats at 1, 5 and 9 days | 146.2 | 3.6 days |
| | rhBMP-2 + NiPAM/NASI | Implantation | 200 μl. | 6 μg | 3.9 mg/ml. | 2 rats at 1, 5 and 9 days | 190.6 | 3.2 days |
| 2 | rbBMP-2 | Implantation | 200 μl. | 6 μg | 28.7 mg/ml. | 2 rats at 1, 5 and 9 days | 217.0 | 3.1 days |
| | rhBMP-2 + NiPAM | Implantation | 200 μl. | 6 μg | 28.7 mg/ml. | 2 rats at 1, 5 and 9 days | 290.2 | 3.8 days |
| | rhBMP-2 + NiPAM/EMA | Implantation | 200 μl. | 6 μg | 28.7 mg/ml. | 2 rats at 1, 5 and 9 days | 233.6 | 3.0 days |
| | rhBMP-2 + NiPAM/NASI | Implantation | 200 μl. | 6 μg | 28.7 mg/ml | 2 rats at 1, 5 and 9 days | 315.6 | 3.8 days |
| 3 | rhBMP-2 | Injection | 100 μl. | 6 μg | 28.7 mg/ml | 2 rats at 1, 5 and 9 days | 72.8 | 1.6 days |
| | rhBMP-2 + NiPAM | Injection | 100 μl. | 6 μg | 28.7 mg/ml | 2 rats at 1, 5 and 9 days | 93.2 | 1.7 days |
| | rhBMP-2 + NiPAM/EMA | Injection | 100 μl. | 6 μg | 28.7 mg/ml. | 2 rats at 1, 5 and 9 days | 473.4 | 4.6 days |
| | rhBMP-2 + NiPAM/NASI | Injection | 100 μl. | 6 μg | 28.7 mg/ml | 2 rats at 1, 5 and 9 days | 419.2 | 4.4 days |

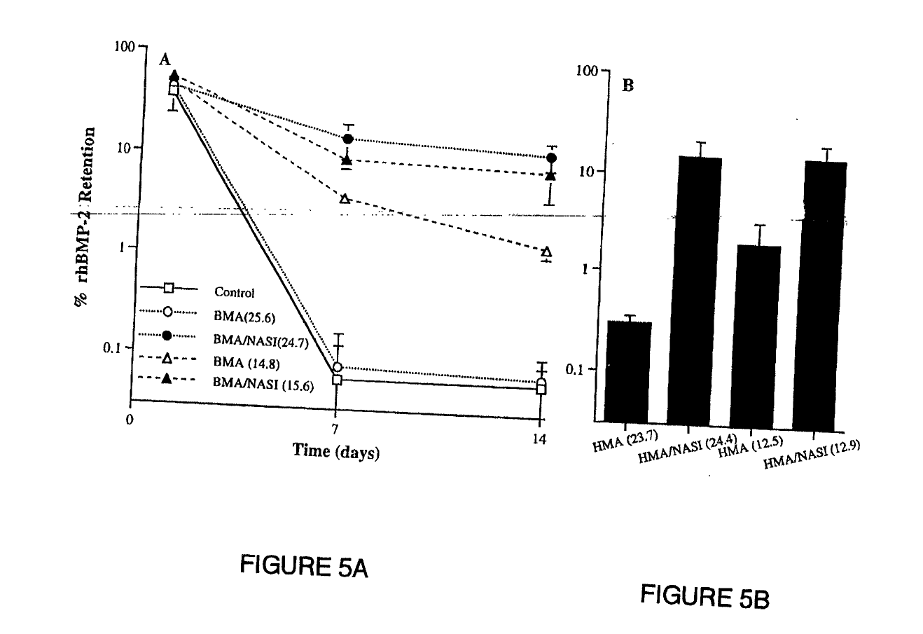

For all PK studies, blood samples were taken by cardiac puncture and femur and tibiae were routinely harvested. There was no radioactivity in any of the harvested organs. Only urine exhibited a high level of radioactivity, consistent with the expected degradation pathway of the radiolabeled rhBMP-2. Autoradiography showed that the highest radio intensity was observed in rhBMP-2 injections with NiPAM/NASI and NiPAM/EMA on day 1, and 5 (FIG. 5). Only a trace of radiointensity remained in control rhBMP-2 NiPAM groups on day 5. The distribution of ¹²⁵I-labeled rhBMP-2 in the muscle seemed to be spread to whole muscle compartment for NiPAM/EMA but was more confined around the injected site at the center of gluteus maximus for NiPAM/NASI.

### EXAMPLE 6

In Vitro Release

The polymer solutions prepared for in vivo studies were also used for in vitro assessment of rhBMP-2 release. When release from Helistat® sponges was determined, 200 μL radioactive rhBMP-2 solution was soaked into a sponge which was then placed in a test tube. One mL of SBF was added to the test tubes and incubated at 37° C. In the case where release without sponges was determined, 100 μL of rhBMP-2 solution was added to the bottom of a test tube, the temperature was raised to 37° C. to induce polymer gelation and 1 mL of SBF was added to the test tubes. The SBF was periodically exchanged after centrifugation at 500 g for 8 minutes. The radioactivity in the supernatant was counted. The rhBMP-2 retention was calculated by: {(cpm−cpm₁)=cpm₁}×100%, where cpm₁=initial counts and cpm¹=counts released into SBF at time t.

A slow release of rhBMP-2 from the collagen sponge was observed in vitro (FIG. 2A). Approximately 50% of rhBMP-2 was retained in the sponge after 72 hours. There was a slight (8-15%) decrease in initial rhBMP-2 retention when NiPAM, NiPAM/EMA and NiPAM/NASI were added to rhBMP-2 solution at 3.9 mg/mL. The subsequent retention profiles were not significantly different with or without the polymers. At a higher polymer concentration of 28.7 mg/mL, the retention profiles did not significantly change (not shown). When release from the gelled polymer was assessed in the absence of a sponge (FIG. 2B), NiPAM/NASI retained a higher level of rhBMP-2 up to 72 hours after which a significant drop in retention was noted. The time course of retention among the other polymers was similar in the latter case. Note that the release of control rhBMP-2 without any polymer was not complete (i.e., retention >0%), indicating relative insolubility of rhBMP-2 in the SBF medium.

### EXAMPLE 7

Statistical Analysis

Where indicated, one-way ANOVA with LSD posthoc multiple comparison programs (STATISTICA; StatSoft Inc., Tulsa, Okla.) were used for statistic analysis (p<0.05). A variation of >20% between two PK parameters was considered significant [Ritschel Meth. Find. Exp. Clin. Pharmacol (1992) 14:469-482]. The latter statistical measure is used to investigate the bioequivalence of pharmaceutical formulations.

Based on the examples desribed above, the polymer LCST was considered to be a critical parameter for drug delivery application in vivo. It needs to be lower than the physiological temperature of 37° C. and the difference between the polymer LCST and the physiological temperature is expected to determine the polymer dissolution rate in vivo. For a polymer designed to physically entrap a protein, this difference may ultimately determine the protein release rate. The LCST for NiPAM in for examples above was −27° C. (in phosphate buffer), lower than the commonly reported LCST of 30-33° C. (in water) [Schild Water Soluble Polymers: Synthesis, Solution Properties, and applications (1991) ACS press Washington D.C. p. 249]. The difference is likely due to buffer composition in which the polymer was dissolved. To determine whether LCST is critical for rhBMP-2 delivery, EMA were incorporated units into the NiPAM polymers and demonstrated a significant LCST decrease. It has been shown that the reduction in LCST was proportional to the EMA mole % of the polymer and for the hydrophobicity of the polymer was additionally confirmed by the polymer film dissolution study Fan and Uladag Drug Delivery in the 21st Century (2000) ACS Washington D.C. One other property observed with the NiPAM/EMA copolymer was its ability to form a gel when the solution, where a temperature increase resulted in typical micellar formation but formation of a semi-stable gel NiPAM/EMA did not exhibit increased retention of rhBMP-2 in implant study. A combination of lower LCST and propensity for gelation were the likely reasons for better rhBMP-2 retention by NiPAM/EMA.

Additional engineering performed with the thermoreversible polymers was directed to the inclusion of protein reactive NASI groups into the NiPAM backbone. rhBMP-2 conjugation to the NiPAM/NASI was achieved by simply mixing the two in a medium devoid of amines. NASI also acted as a hydrophobic unit effectively lowering the LCST (more so than the EMA based on per unit monomer incorporated into the polymer). The NiPAM/NASI films were not stable and did not undergo gelation in the phosphate buffer in vitro. A hydrolysis of NASI groups, which yields negatively charged carboxyl groups and increases polymer solubility, was possibly responsible for buffer and incubated in SBF (i.e., during in vitro release studies). NASI reaction with either the protein or the components of glycine buffer appear to stabilize the polymer gel after 3 days time the gels began to dissolve and rhBMP-2 was released, suggesting polymer hydrolysis as a release mechanism. These polymers were effective in retaining rhBMP-2 in an implantable format at a high concentration, and especially in an injectable format. A NiPAM/NASi gel was present at the administration site in vivo even after 9 days, indicating that polymer was stable gel formation in vivo. Expected to be based on the additional NiPAm/NASI reaction with components of interstitial fluid or extracellular matrix proteins. Should NiPAm/NASI have reacted with multifunctional amines such as endogeneous proteins, this might have resulted in a stable crosslinked network in vivo.

There was not much difference in the initial (day 1) rhBMP-2 retention in either implantable or injectable delivery mode (40% and 56% in two implant studies, and 30% in the injection study). The presence of the collagen sponge did not appear to be significant in initial retention in our intramuscular model. The rhBMP-2 loss in a mouse subcutaneous injection model was much faster: >99% release in a day [Bromberg and Ron Adv. Drug Del. Rev. (1998) 31: 1997-221]. Visual observation in intramuscular injection model indicated retention of injected fluid among the muscle fibers, which apparently hold the injected rhBMP-2 better than a subcutaneous site where no cavity is available for fluid retention. The subsequent release was much faster without the collagen sponge, whose primary function appeared to be slowing the rhBMP-2 loss from an administration site. Two polymers, NiPAM/EMA and NiPAM/NASI, were even more effective in the absence of the collagen sponge (compare day 9 rhBMP-2 retention data between FIG. 2 and FIG. 3). It is possible that the sponge interfered with polymer-polymer interaction necessary to form a stable gel in vivo.

The three polymers used in this study did not adversely affect the rhBMP-calcium incorporation into the implants. Histological assessment of de novo bone deposition was not dependent whether the polymers were implanted with or without the biomaterial. A physical entrapment (NiPAM/EMA polymers) as well as a chemical conjugation (NiPAM/NASI) mechanism appear to be equally effective. Better rhBMp-2 retention should ultimately result in a more potent osteoinduction. Based on the present invention, in which engineered biomaterials are included in conventional rhBMP-2 formulations, provides an alternative to current approaches to control in situ BMP levels. The latter relies on a scaffold's ability to retain the protein after being wetted with the protein solution. The scaffold, in addition to protein retention, is expected to exhibit a spectrum of properties for optimal osteoinduction. By relying on thermoreversible polymers for rhBMP-2 retention, it may be possible to engineer a scaffold independent on its properties responsible for rhBMP-2 retention.

### EXAMPLE 8

Effects of Molecular Weight of Thermoreversible Polymer on In Vivo Retention of BMP-2

A. Polymer Properties

From a range of polymers, four polymers were chosen for this study and the compositions of these polymers were shown in Table 2. Compared to the feed ratios, the final EMA content was increased by 5-6% in polymers A and B, and by 11-12% in polymers C and D, irrespective of the presence of NASI in the polymerization mixture. NASI content in polymers were typically ˜50% of the feed ratios in either polymerization scheme. The primary reason for the choice of these polymers was the similarity in LCST (all polymers exhibited an LCST of 20-22° C.), but a large variation in their MWs. The polymers synthesized by BPO/ter-butyl alcohol scheme were approximately 8.5 times larger than the polymers from V-501/dioxane scheme.

TABLE 2
Composition, LCST and MW of thermoreversible polymers used in this study

| | | | | |
|---|---|---|---|---|
| Polymer | Monomer Feed Ratio (%) | Polymer Composition (%) | LCST | MW |

| (assigned Code) | NiPAM | EMA | NASI | NiPAM | EMA | NASI | (° C.) | (kD) |
|---|---|---|---|---|---|---|---|---|
| NiPAM/EMA (A) | 90.0 | 10.0 | 0.0 | 84.8 | 15.2 | 0.0 | 22.0 | 48.0 |
| NiPAM/EMA/NASI (B) | 87.0 | 10.0 | 3.0 | 82.1 | 16.3 | 1.6 | 20.2 | 49.8 |
| NiPAM/EMA (C) | 84.6 | 15.4 | 0.0 | 73.7 | 26.3 | 0.0 | 20.3 | 404.0 |
| NiPAM/EMA/NASI (D) | 83.0 | 15.1 | 1.9 | 71.8 | 27.2 | 1.0 | 21.5 | 422.0 |

B. Structure of Polymer Hydrogels

Water uptake of the gels showed a significant difference between the polymers synthesized by different polymerization schemes. The hydrogels from polymers A and B have a higher water uptake than the hydrogels from polymers C and D. The difference was evident after 1 and 12 hour of hydrogel formation. The presence of NASI in polymers did not affect the water uptake. All hydrogels demonstrated a porous micelle with different shapes and orientations. A longitudinal cell was mostly seen in hydrogels of A and B, and a square or round chamber in hydrogels of C and D. The largest diameter of the cell was present in polymer B, and then in polymers A, D and C in a descending order, but the thickness of cell wall was in reverse order for these polymers at 3 hours. The porosity of the hydrogels underwent a dramatic decrease in polymers C and D (approximately 20- and 6-fold, respectively) more so than the polymers A and B (approximately 3-4 fold), as a result of 12 hour incubation at 37° C. (FIG. 2). Although the presence of NASI did not result in an appreciable difference in morphology for low MW polymers (comparing B with A), the presence of NASI in high MW polymers (comparing C with D) resulted in larger pores after 12 hours incubation.

C. In Vivo Reactivity and In Vivo Retention of rhBMP-2

The chosen polymers were formulated with rhBMP-2 at 4° C. as an injectable solution and directly injected intramuscularly to assess rhBMP-2 retention at the application site. The retention was assessed after 14 days since our previous results indicated this time-point to be representative of the relevant release duration [J. Biomed. Mat. Res. 50:227-238 (2000)]. Polymer C sequestered the highest fraction of rhBMP-2 in the injected muscle compartment. The difference was 2.1-, 2.7- and 108-fold compared to the polymers D, B and A, respectively. The rhBMP-2 retention by polymer A was insignificant and comparable to rhBMP-2 injection alone without any carriers. Polymers containing protein-reactive group (NASI) gave an equivalent rhBMP-2 retention irrespective of MW (comparing B with D). Autoradiography of the explanted muscle tissue also indicated a superior retention of rhBMP-2 by polymer C, followed by polymers D and B and finally by polymer A.

High MW polymers formed a more compact, or hydrophobic gel in phosphate buffer at 37° C. as compared to low MW polymers. Correspondingly, high MW NiPAM/EMA polymer (C, 404 kD) demonstrated a higher rhBMP-2 retention in vivo (p<0.001) as compared to low MW NiPAM/EMA (A, 48 kD). The differences in pore size shift between 3 and 12 hrs disclosed by SEM implied a possible reason for varied rhBMP-2 entrapment between the two polymers. The NiPAM/EMA polymer with high MW formed a stable gel with the average pore size much smaller than that in the polymer with low after injection into the body temperature. The smaller pore size is likely to prevent the initial burst release of rhBMP-2 entrapped from the polymer gel at 37° C. The pore size was further declined ˜20 times for high MW polymer instead of only 3-4 times for low MW polymer after 12 hrs. The former polymer retained rhBMP-2 more efficiently in a dense micelle of the gel. The remnants of the high MW polymer gel still existed in the muscle compartment when the specimens were retrieved while the low MW polymer gel was totally disappeared on day 14. This observation indicated that the kinetics of swelling/dissolution of the polymer-rhBMP-2 preparation was markedly affected by the MW of the polymers [Pharm. Res. 819-827 (1999)]. The MW of the synthesized polymer influences the stability water uptake of the hydrogel in vitro loading capacity and entrapment of rhBMP-2 in vivo. For polymers containing no protein-reactive group, the LCST and MW of synthesized polymers are two determinant factors for rhBMP-2 delivery in vivo.

The NiPAM/EMA/NASI polymer with high MW did not show any superiority of rhBMP-2 reaction in vitro compared to low MW polymers. A significant effect of NASI was evident for the low MW polymers where the rhBMP-2 retention after 14 days was ˜52-fold higher with polymers containing NASI. However, such a NASI effect was not observed with high MW polymers. The presence of NASI appeared to significantly (p<0.006) reduce the rhBMP-2 retention in high MW polymers. Unlike polymers without NASI groups, the performance of NASI-containing polymers did not depend on the polymer MW.

### EXAMPLE 9

In Vivo Studies of BMP-2

A select set of NiPAM/AMA and NiPAM/AMA/NASI polymers were chosen that exhibited either low or high LCST (13-17 vs. 24-26° C.; see FIG. 4 for polymer compositions). The reactivity of the polymers with rhBMP-2 was investigated using SDS-PAGE: a fixed ratio of rhBMP-2 and polymer (1:25 on mass basis) was incubated and the disappearance of native rhBMP-2 band was assessed as a function of time. The spectroscopic method was not used in this case because of the need for large amount of protein (>10 mg) in this set-up. SDS-PAGE analysis indicated that there was no reaction or association between the NiPAM/AMA polymers and rhBMP-2 irrespective of the choice of AMA. With NASI containing polymers, a time-dependent increase in protein conjugation was observed. No significant changes in native rhBMP-2 band was evident after 3 hour of incubation. A significant reduction of native rhBMP-2 band was visible after 6 hours and, by 20 hours, all native rhBMP-2 disappeared at the usual migration band of 33 kD. The protein was detected at higher MWs consistent with rhBMP-2-polymer conjugates. This was further confirmed in immunoblots, which indicated the presence of high MW rhBMP-2 species upon incubation with NiPAM/AMA/NASI, but no changes in rhBMP-2 MW upon incubation with NiPAM/AMA (not shown). Based on the polymer MWs from light scattering studies and the MWs of rhBMP-2 conjugates on SDS-PAGE, multiple polymer chains were apparently conjugated to each rhBMP-2 molecule. SDS-PAGE indicated that all rhBMP-2 is effectively conjugated to NASI-polymers at the chosen protein:polymer ratios. More importantly, the LCST of the NASI-polymers did not affect the conjugation efficiency since all NASI-containing polymers, irrespective of the nature of AMA (EMA, BMA or HMA) or the AMA amount were equally effective in rhBMP-2 conjugation. This result confirmed the possibility of tailoring the LCST of thermosensitive polymers without compromising the protein reactivity.

BMA-based polymers were further evaluated for rhBMP-2 delivery in an intramuscular injection model. The polymers were incubated with rhBMP-2 for 20 hours under similar conditions to the SDS-PAGE study. The rhBMP-2/polymer solutions were then directly injected into the hind legs of rats. A two week study period was utilized since this represented an adequate time period for osteoinduction in the chosen animal model. The polymers were chosen to have a high (˜25° C.) or low (˜15° C.) LCST, and each with and without NASI. The LCST was not likely to change for the injected polymers since protein conjugation was previously shown not to alter the polymer LCST, and the 20-hour incubation period was not long enough to exhibit an LCST elevation in time-course studies. The rhBMP-2 retention was similar on day 1 (37-43%, p>0.12) for rhBMP-2 injection alone and injection with NiPAM/BMA and NiPAM/BMA/NASI polymers that had a high LCST (FIG. 5A). The NiPAM/BMA and NiPAM/BMA/NASI polymers with low LCST retained a significantly higher rhBMP-2 on day 1 (46 and 54%, respectively; p<0.02). Even with the latter polymers, a significant fraction of rhBMP-2 (˜50%) was lost as a burst release, which was likely due to inability of the polymers to rapidly precipitate. By day 7, an insignificant fraction of rhBMP-2 (<0.1%) was retained at the site for rhBMP-2 injection alone as well as injection with high LCST (25.6° C.) NiPAM/BMA. Although injection with low LCST (14.8° C.) NiPAM/BMA gave a higher retention on days 7 and 14, the difference from the rhBMP-2 injection alone was not significantly different (p>0.3). A significantly (p<0.03) higher retention was obtained with NASI-containing polymers (>100-fold difference on days 7 and 14 compared to rhBMP-2 injection alone). A 10° C. difference in LCST for the latter two polymers did not appear to influence the protein retention on day 7 and 14 (p>0.06).

A final study was set-up to extend the results to the NiPAM/HMA and NiPAM/HMA/NASI polymers (FIG. 5B). In this study, the rhBMP-2 was injected with the chosen polymers and rhBMP-2 retention was determined only on day 14. The highest rhBMP-2 retention was again with the NASI containing polymers. A 11.5° C. difference in LCST did not make a significant impact on rhBMP-2 retention for these polymers. The NiPAM/HMA with lower LCST (11.2° C.) gave a 6.2-fold higher rhBMP-2 retention compared to the NiPAM/HMA polymer with higher LCST (23.7° C.; p<0.05). The latter gave an equivalent retention to that of rhBMP-2 injection alone.

These results indicated that thermosensitive polymers whose LCSTs were lower than the LCST of parent NiPAM homopolymer were occasionally effective to retain co-delivered rhBMP-2 (significant difference for NiPAM/BMA on day 1 and NiPAM/HMA on day 14). Polymers capable of chemically conjugating the protein, were more effective for retention. rhBMP-2 retention obtained with implantable, absorbable collagen sponges (ACS) is the clinical choice for the delivery of rhBMP-2 and its rhBMP-2 retention profile is superior to numerous other biomaterials utilized to deliver rhBMP-2 in animal models. The initial retention obtained in this study was comparable to rhBMP-2 retention implanted with ACS: 2 separate studies gave 40-60% retention for implantable rhBMP-2, compared to 37-53% for injectable rhBMP-2 in this study. However, unlike ACS which exhibited a continuous loss of rhBMP-2 for 2-weeks, the rhBMP-2 loss from thermosensitive polymers was relatively small between the first and second week of study. This resulted in >10-fold better retention at the end of 2 weeks (˜10% in this study vs. 0.5-1% by ACS implants). These results indicated the possibility of developing injectable rhBMP-2 formulations using thermosensitive polymers that retains the proteins equivalent or even superior to clinically used implantable formulations.

The foregoing descriptions detail presently preferred embodiments of the present invention. Numerous modifications and variations in practice thereof are expected to occur to those skilled in the art upon consideration of these descriptions. Those modifications and variations are believed to be encompassed within the claims appended hereto.

What is claimed is:

1. A composition for delivery of osteoinductive proteins comprising a temperature-sensitive polymer.

2. A composition for delivery of osteoinductive proteins said composition comprising
a) osteoinductive protein; and
b) temperature sensitive polymer.

3. The composition of claim 2 further comprising a carrier.

4. The composition of claim 2 wherein the osteogenic protein is selected from the group consisting of members of the BMP family.

5. The composition of claim 4 wherein the osteogenic protein is BMP-2.

6. The composition of claim 1 wherein he temperature sensitive polymer comprises

7. The composition of claim 3 wherein the carrier is a collagen derivative.

8. A composition for delivery of osteogenic proteins admixture comprising.
a) BMP-2
b) a temperature sensitive polymer; and
c) a collagen sponge carrier.

9. The composition of claim 1 wherein the osteogenic protein is BMP-2.

10. A method for inducing the formulation of bone comprising administering to a patient in need of same a composition comprising an osteoinductive protein and a temperature-sensitive polymer.

11. A composition for retention of therapeutic proteins at an application site said composition comprising a thermo-reversible polymer and a therapeutic protein.

12. A method for retention of therapeutic proteins at an application site said method comprising administering a composition comprising a thermoreversible polymer and a therapeutic protein.
